(19) United States
(12) Patent Application Publication
(10) Pub. No.: US 2012/0237405 A1
(43) Pub. Date: Sep. 20, 2012

(54) Flatch Evac

(76) Inventors: John Wesley King, West Helena, AR (US); Shelley Kay Haynes, West Helena, AR (US)

(21) Appl. No.: 13/047,006

(22) Filed: Mar. 14, 2011

(51) Int. Cl. A61L 9/00

(52) U.S. Cl. 422/120

(57) ABSTRACT

The objective of the invention is to provide an odor neutralizer that targets the source of odor-causing molecules, capturing and channeling them to a filter, and preventing escape of odor. Current technology does not target the odor source, and odor-causing molecules escape into uncontaminated air or are mixed with other smells, causing unpleasantness and/or embarrassment or even indignity. Targeting the odor source, my invention, the Flatch Evac, is designed to eliminate odors and harmful gases at the source.

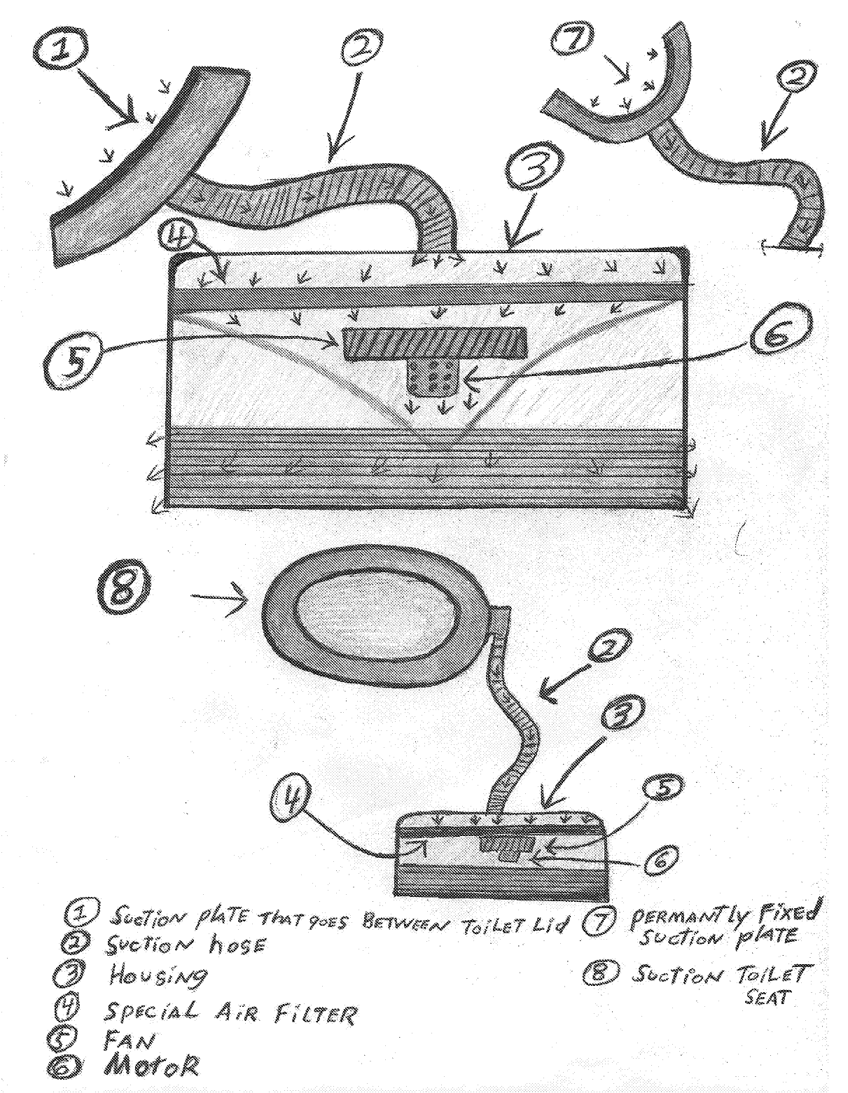

The present invention relates to a motorized odor neutralizer that takes in and filters odorous molecules at the source.

The device is comprised of the following:
The components consist of an adjustable intake port; detachable, curved air intake duct with flexible rubber seal; motor and filter housing unit made of a durable, rigid material ; at least one chemical air filter; electrical motor; suction fan; switch; and power source. All components listed as part of my invention include but are not limited to the materials of which they are comprised. Measurements of parts and voltages for the aforementioned components may vary depending on the desired purpose of the device.

My invention works to benefit society by providing a means by which a person can live and/or die with dignity, being free from the worry of unpleasant odors, which include but are not limited to bodily emissions. The materials used to construct this invention may be varied according to its particular situational intent for optimal performance. The efficiency of this invention is dependent upon its ability to capture odor-causing molecules at the source.

Any connections in this application may include any known adhesives or tools.

The air intake duct is at the superior end of the device, with rubber seals attached to the interior top and bottom lip, conforming to the shape of the object to which it is affixed. The adjustable intake port(s) attaches to penetrate the air intake duct at one end and the motor/filter housing at the other. The chemical air filter(s) is situated in the air flow path of the intake duct and port. The suction fan is attached to the motor, located inside the housing unit. The power source connects to the motor, and the switch opens or closes the electrical path for power. The connections of the components of this device may be inverted or rearranged depending on the schematics around which the device may be designed.

The air intake duct is open intermittently between rubber seals facing the source(s) of odor. The suction fan attached to the motor pulls contaminated air through the intake duct and intake port toward the chemical air filter, where the odor-causing molecules are neutralized.

The invention can be made by using a flexible hose as an air intake duct, with slots to face the odor-causing molecules. A durable pipe or tube may be used as the air intake port by inserting it into the air intake duct at one end and the housing unit at the other. Inside the housing unit, a chemical air filter is attached to a suction fan and motor, with the filter situated in the path of incoming contaminated air, thereby neutralizing the odor.

The elements could be reconfigured in a variety of ways depending on intended use of the device. The invention is intended to be versatile in ways that include but are not limited to size, shape, capacity, and portability.

The use of this invention would be to eliminate odor-causing molecules at the source(s). It could be used in bathrooms, conforming to or as part of toilet seats, or in facilities that include but are not limited to hospitals, hospices, morgues, and waste industries. The air intake duct would face and be placed at, or as closely as safely possible to, the odor source pulling the contaminated air into the housing for odor neutralization.

This invention can be used in individuals' households as well as commercially. It can be modified for various uses in industries including but not limited to medical supplies. My invention can be portable or stationary. It can be used in any situation that requires the technology associated with it.

The current technique for neutralizing odors does not target the source of odor-causing molecules, or it uses “fresheners” that simply mix with the odor; but my invention targets the source of odor-causing molecules.

The existing air ventilators pull contaminated air through a space so large that too many odor-causing molecules escape capture, allowing odor to escape. The nature of a gas causes it to fill its container, whatever the size. The unpleasantness is experienced by any olfactory cells present. Mixing air fresheners with an odor just adds another smell to the mix. My invention targets the odor source, thereby reducing odor-causing molecule escape.

What is claimed is:

1. My invention, the Flatch Evac, is an odor neutralizer that targets odor-causing molecules at their source(s), preventing odor escape into uncontaminated air.
